(19) United States
(12) Patent Application Publication
(10) Pub. No.: US 2009/0325214 A1
(43) Pub. Date: Dec. 31, 2009

(54) NUCLEAR MATRIX PROTEIN ALTERATIONS ASSOCIATED WITH COLON CANCER AND THEIR USE AS MARKERS FOR COLORECTAL CANCER

(75) Inventor: Robert H. GETZENBERG, Lutherville, MD (US)

Correspondence Address: FOLEY AND LARDNER LLP;SUITE 500, 3000 K STREET NW, WASHINGTON, DC, 20007, US

(73) Assignee: University of Pittsburgh - Of the Commonwealth System of Higher Education

(21) Appl. No.: 12/470,576

(22) Filed: May 22, 2009

Related U.S. Application Data
(60) Provisional application No. 61/071,895, filed on May 23, 2008.

(51) Int. Cl. C12Q 1/02

(52) U.S. Cl. 435/29

(57) ABSTRACT

CCSA-2 levels in serum samples from individuals suffering from colorectal cancer can be used gauging the progression of a colorectal cancer condition. In particular, the progression of a colorectal cancer condition is gauged by comparing the level of serum CCSA-2 from individuals having colorectal cancer to predetermined CCSA-2 levels that correlate to the state of advancement of a cancer condition. Additionally, serum CCSA-2 levels can be used by a clinician to gauge the efficacy of a colorectal cancer treatment.

### CROSS-REFERENCE TO RELATED PATENT APPLICATIONS

This application claims priority from U.S. Provisional Application Ser. No. 61/071,895, filed May 23, 2008, incorporated herein by reference in its entirety.

### STATEMENT REGARDING FEDERALLY SPONSORED RESEARCH

This invention was partially funded by CA084968, awarded by the National Cancer Institute. The United States government has certain rights to this invention.

### BACKGROUND OF THE INVENTION

The present invention relates generally to the field of proteins that are associated with proliferative cellular diseases such as cancer, and in particular to colon and rectal cancers. In particular, the invention relates to a specific biomarker for detecting colon cancer in an individual.

Colon cancer is the third most common form of cancer diagnosed worldwide. Etiologically, colon cancer involves an adenoma-carcinoma sequence that requires the evolution of an adenoma to a carcinoma. Adenomas are generally benign growths (polyps), having cells of glandular origin. However, over time the polyps may grow and become malignant, so as to form a carcinoma. Thus, individuals with adenomatous polyps have a higher risk of incidence for colon cancer.

Traditional methods for detecting colon cancer have involved fecal occult blood testing, flexible sigmoidoscopy, contrast enema, and colonoscopy. However, each method has its own drawbacks, such as the lack of sensitivity, specificity, or risk of complications and discomfort due to the invasive nature of the screen. Thus, while the aforementioned methods have resulted in lowering the mortality rate of colon cancer patients, there still exists a need for identifying non-invasive methods based on highly specific biomarkers that can be used for detecting and following the progression of a colorectal cancer condition.

The present invention fills this niche, by providing a sensitive and highly specific serum-based biomarker for detecting colon cancer. In particular, the present invention allows the identification of individuals having a neoplastic lesion, from those that do not have a neoplastic lesion. Accordingly, a method capable of separating patients with advanced adenomas and colorectal cancer from those patients displaying heperplasticity or having non-advanced adenomas, or other types of cancer conditions is provided. Also disclosed, is a method for gauging the advancement of a colorectal cancer condition.

The information provided herein is solely to assist the understanding of the reader. Neither this information nor any publication cited is admitted to be prior art.

### SUMMARY OF THE INVENTION

The present invention provides a method for gauging the state of advancement of a colorectal cancer. The inventive methodology involves obtaining a sample of blood from an individual suffering from colorectal cancer and analyzing the sample for the level of a colon cancer specific antigen, CCSA-2. The level CCSA-2 in the sample is then correlated to at least one value that is predetermined for gauging the advancement of a colorectal cancer.

In another aspect, the inventive methodology involves correlating CCSA-2 levels from individuals having colorectal cancer to the CCSA-2 levels in non-cancerous tissue of the same individual or to CCSA-2 levels from one or more persons other than the individual having cancer.

Further this aspect of the invention, the correlating step identifies an individual with an advanced adenoma prior to malignant transformation.

Another embodiment of the present invention provides a method for gauging the efficacy of treatment for a colorectal cancer condition. The claimed methodology involves obtaining at least two samples at different time points during the treatment of a subject. CCSA-2 levels are determined in the samples and the levels of CCSA-2 in the two samples are compared. Efficacy is gauged from the difference in the levels of CCSA-2 between the two samples. According to the claimed method, a lower level of CCSA-2 in the sample obtained at a later time point, during treatment, indicates a good efficacy of treatment.

### BRIEF DESCRIPTION OF THE DRAWINGS

FIG. 1 is a graph showing the specificity of CCSA-2 antibodies. As shown in the figure, the CCSA-2 antibodies are highly specific with no cross-reactivity to related colon cancer proteins CCSA-3 and CCSA-4, as well as to early prostate cancer antigen (EPCA) and early prostate cancer antigen-2 (EPCA-2).

FIG. 2 is a graph that shows the ability of CCSA-2 to distinguish normal, hyperplastic and benign individuals from individuals having colon cancer. A serum based indirect ELISA assay was used for diagnosis. As shown in the figure, a CCSA-2 concentration of ≧10.8 μg/ml is the cut-off concentration for separating normal individuals from individuals having colon cancer.

FIG. 3 is a ROC curve for CCSA-2. The figure correlates the specificity of antibodies for CCSA-2 to the sensitivity of the assay based on CCSA-2 concentrations in the sample. As shown in the figure, CCSA-2 can be used as a biomarker to separate normal, hyperplastic and benign individuals from individuals with cancer.

FIG. 4 is a ROC curve showing that CCSA-2 is a good biomarker for separating non-advanced adenoma individuals from individuals with advanced adenoma.

### DETAILED DESCRIPTION OF THE PREFERRED EMBODIMENTS

The present invention comprehends a non-invasive method for gauging the progression of colorectal cancer, based on the level of a colon cancer specific protein CCSA-2, in the serum of individuals suffering from colon cancer. See Getzenburg et al., Clin. Cancer Res. (2008), 14(5), 1349-1354. CCSA-2 is a nuclear matrix protein that has been identified as a specific biomarker for colon cancer. In the context of the present application, the phrase “nuclear matrix protein” refers to proteins that are part of a 3-dimensional filamentous protein network that provide the necessary framework for maintaining the overall size and shape of the nucleus. The present invention also allows distinguishing individuals with advanced adenomas from those with non-advanced adenomas or hyperplastic lesions.

A hallmark of cancerous cells, are structural changes in the shape and size of the cells nucleus. Accompanying these morphological changes, are changes in the composition and expression levels of nuclear matrix proteins (NMP's). Analysis of the NMP's from cancer cells revealed, that some nuclear matrix proteins exhibit different physicochemical properties when compared to the corresponding protein in normal cells. These results indicate that specific NMP's can be used as biomarkers for a cancer condition.

Direct support for the role of a nuclear matrix protein as a biomarker for colorectal cancer comes from the discovery, that CCSA-2 is a specific biomarker for colorectal cancer. In particular, the present inventor found that serum CCSA-2 levels can be used to distinguish normal individuals from individuals having colorectal cancer. Pursuant to one embodiment of the present invention, therefore, the serum level of CCSA-2 protein is a reliable marker for a colorectal cancer condition. In accordance with this aspect, CCSA-2 levels are higher in an individual having colorectal cancer than in individuals with hyperplastic lesions, non-advanced adenomas or advanced adenomas.

Another aspect of the present invention relates to separating individuals with advanced adenomas from those with non-advanced adenomas, based on their serum CCSA-2 levels. Thus, the present invention contemplates using serum CCSA-2 levels as a risk stratification marker for distinguishing individuals with a high risk for developing colorectal cancer (advanced adenomas ), from those having a lower risk for developing colorectal cancer (non-advanced adenomas).

In the context of the present invention, the term “advanced adenoma” refers to an adenoma polyp having a size of 1 cm or greater or an adenoma with villous components, or with a high-grade as determined histologically or severe dysplasia. The term “non-advanced adenoma” refers to an adenoma polyp having a size less than 1 cm. Furthermore, in context of the present invention, the term “risk stratification” refers to a statistical process for separating individuals with a high pre-disposition for colorectal cancer (high risk ) from those with a lower pre-disposition for colorectal cancer (low risk).

The detection of CCSA-2 in serum is not limited to one specific analytical method. For example, any conventional method, such as gel electrophoresis, mass spectrometry, high performance liquid chromatography or immunoassay can be used for the detection of CCSA-2 protein in serum. While each method has its own advantages and drawbacks, immunochemical methods are often preferred in clinical settings. Some of the advantages for using an immunochemical method such as an ELISA, for clinical applications are speed, specificity, sensitivity and ease with which results can be obtained using this analytical method.

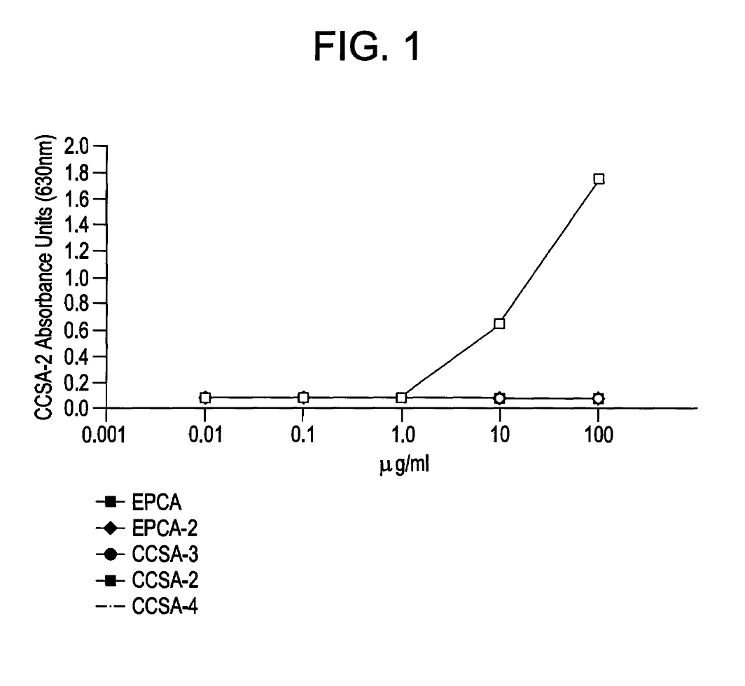

In one aspect, the present invention uses an indirect ELISA for detecting CCSA-2 in serum. According to this immunochemical method, antibodies to CCSA-2 are used to specifically bind the protein, and the antibody-protein complex is then detected using a secondary antibody probe (e.g., an enzyme linked anti-CCSA-2 antibody). The primary antibodies used in the CCSA-2 assay are obtained using a peptide whose sequence was derived from a CCSA-2 epitope. The specificity of a CCSA-2 antibody was further evaluated in serum that contains various cancer cell specific proteins. As shown in FIG. 1, CCSA-2 antibodies are highly specific and display no cross-reactivity to other cancer related proteins, including the colon cancer specific proteins CCSA-3 and CCSA-4. See U.S. Pat. No. 7,189,823. This result indicates that antibodies to CCSA-2 are highly specific and can be used to detect as well as quantify the presence of this biomarker in serum.

In the present context, an “antibody” is a protein that is made up of one or more polypeptides, substantially encoded by immunoglobulin genes or fragments of such genes. Furthermore, an “epitope” refers to a purified or a partially purified preparation of the protein of interest, or fragments thereof that are suitable for eliciting specific antibodies.

Of particular interest to the inventor is a method for gauging the state of advancement of a colorectal cancer condition, preferably by a serum-based CCSA-2 assay. While earlier studies published by the inventor, had indicated that CCSA-3 and CCSA-4 could be used as biomarkers for identifying a colorectal cancer condition, no documented evidence exists for correlating the serum level of these two proteins to the state of advancement of a colorectal cancer condition. See Getzenburg et al., Cancer Res. (2002), 62, 2437-2442. In contrast to the above, a strong correlation exists between the level of CCSA-2 in serum and the advancement of a colorectal cancer condition. Thus, serum CCSA-2 levels are diagnostic for gauging the advancement of a colorectal cancer condition.

In the context of the present invention, the phrases “advancement of a colorectal cancer condition” and “progression of a colorectal cancer condition” are used interchangeably and refer to an increase in the size of a tumor or spread of cancer in the body.

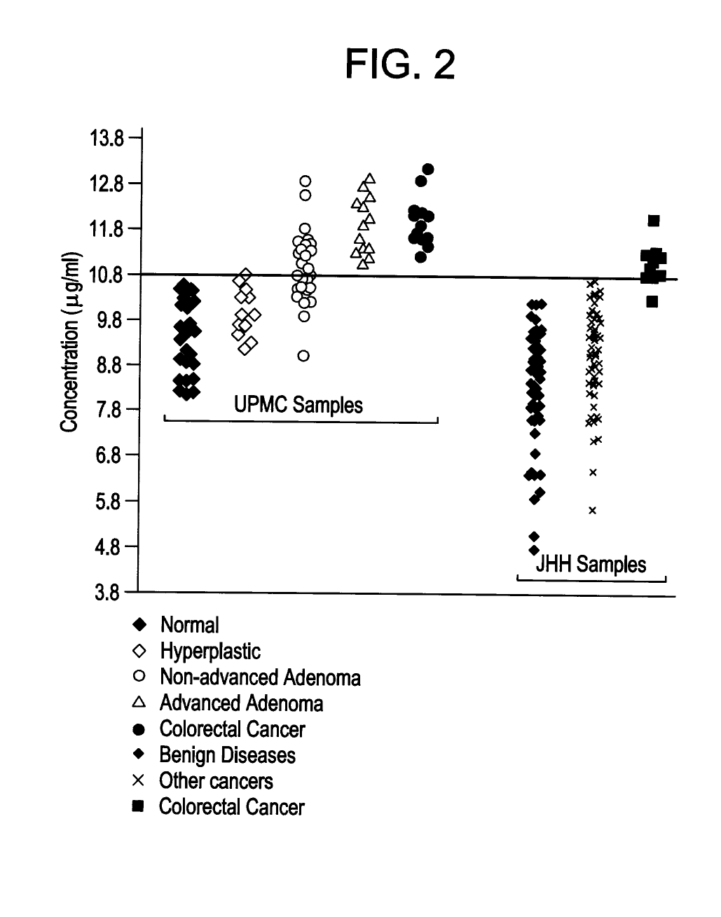

As detailed below, the inventive method was used in a study involving human patients. As shown in FIG. 2, this serum-based approach is highly sensitive and can be used to separate individuals with non-cancerous lesions (e.g., normal individuals or individuals with hyperplastic or benign polyps) from individuals having neoplastic or potentially neoplastic lesions (e.g., colorectal cancer or advanced adenomas respectively). Furthermore, individuals with non-advanced adenomas can be separated from those having advanced adenomas based on the serum CCSA-2 levels of these individuals. In accordance with this aspect of the invention, therefore, a clinician can prescribe a more aggressive treatment regimen for individuals at a higher risk for developing colorectal cancer (advanced adenomas), than those with a lower risk for colorectal cancer.

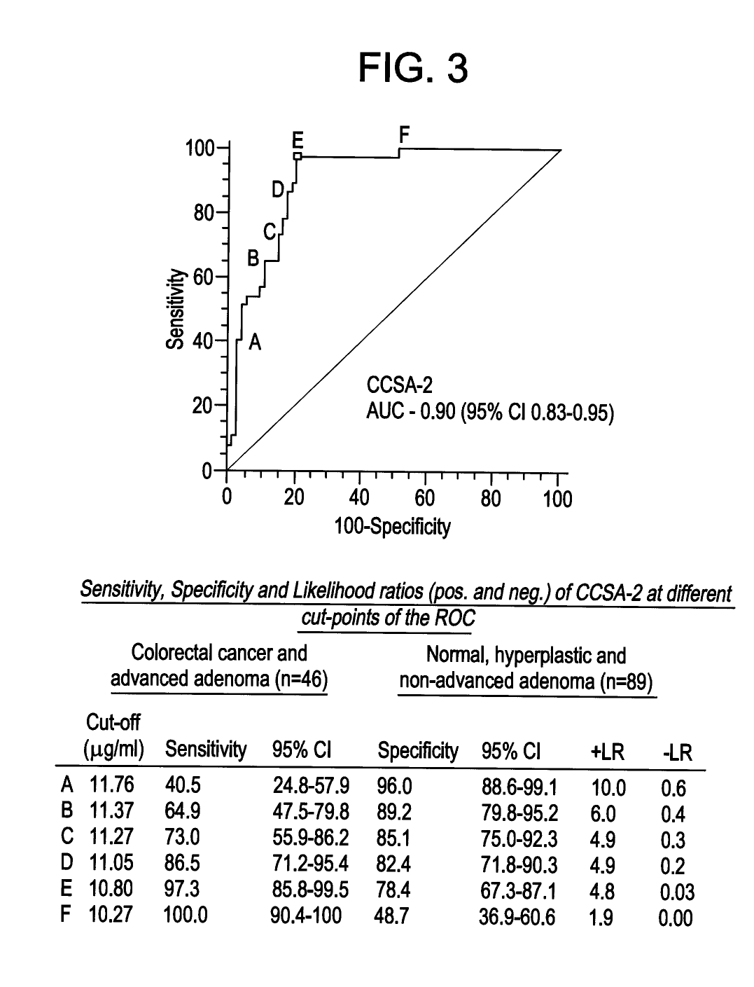

As mentioned above, and shown by the plot in FIG. 3 the inventive methodology is both sensitive and specific for gauging the state of advancement of a colorectal cancer condition using serum CCSA-2 levels. The receiver operating characteristic (ROC) curve shown in FIG. 3 is a plot that depicts the trade-off between sensitivity and specificity for the inventive method, at different cut-off values for serum CCSA-2. In one embodiment, the inventive methodology had a sensitivity of 95.6%, (95% CI)), for detecting individuals having colorectal cancer at a cut-off level for serum CCSA-2≧10.8 μg/ml. Additionally, at the same cut-off value, the inventive methodology showed a specificity (97.7%), separating individuals having colorectal cancer and advanced adenomas from those with benign polyps, or other types of cancers.

In the context of the present invention, the term “sensitivity” refers to a statistical measure of how well the test correctly identifies a particular condition (i.e., the proportion of true positives). The term “specificity” refers to a statistical measure of how well the test correctly identifies negative cases or cases that do not meet a condition under study (true negatives).

Another aspect of the inventive methodology is correlation between serum CCSA-2 levels and the size of an adenomatous polyp. As mentioned above, the development of colorectal cancer involves pathological transformations along the adenoma-carcinoma pathway. While most colorectal cancers originate as small benign adenomatous polyps, malignant transformations leading to the development of a colorectal cancer, are often accompanied by an increase in the size of an adenomatous polyp. While serum levels of previously documented colon cancer specific proteins, CCSA-3 and CCSA-4, had shown no correlation to the size of adenomatous polyps, the present inventor found that there is a significant correlation between the size of an adenomatous polyp and the levels of CCSA-2 in the serum of these individuals.

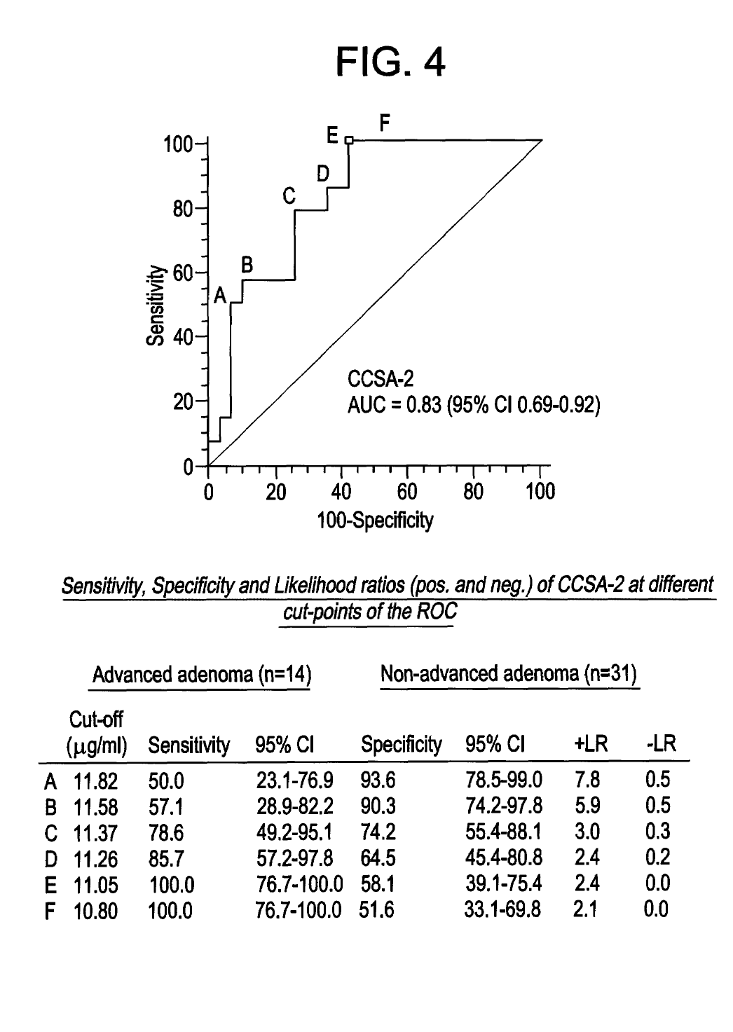

Pursuant to this aspect, individuals having advanced adenomas or cariconmas have higher CCSA-2 levels than individuals with non-advanced adenomas or other forms of benign polyps or cancers. In accordance with the present invention, therefore, serum CCSA-2 levels can be used clinically for gauging the size of an adenomatous polyp. Further support for the role of CCSA-2 levels as a marker for separating individuals with large polyps from individuals having smaller sized polyps comes from Spearman's analysis and the ROC curve for CCSA-2, shown in FIG. 4. The ROC curve has an area of 0.83 (95% CI, 0.69-0.92), and shows that the inventive methodology at a cut-off level for serum CCSA-2≧10.8 μg/ml is both sensitive (97.3) and specific (78.4) for separating individuals with larger polyps (advanced adenomas) from those with smaller polyps (non-advanced adenomas and hyperplastic lesions). These results indicate that serum CCSA-2 levels are diagnostic for gauging the progression of a colorectal cancer condition.

In still another embodiment, the present invention contemplates gauging the efficacy of a treatment regimen for colorectal cancer. Accordingly, the present invention provides a method for gauging therapeutic efficacy, by comparing the levels of serum CCSA-2 in two samples obtained at two different points during treatment from a individual having colorectal cancer. Efficacy of the treatment regiment is gauged from the difference in the levels of CCSA-2 between the two samples. Thus, a lower level of CCSA-2 in the sample obtained at a later time point, during a treatment regimen, indicates good therapeutic efficacy. Methods pursuant to the present invention, therefore, would permit a clinician to monitor the efficacy of a treatment regimen, and alter treatment, if required, so as to improve the therapeutic efficacy of drugs used.

What is claimed:

1. A method for gauging the state of advancement of a colorectal cancer, comprising: (A) providing a blood sample from an individual suffering from said cancer; (B) analyzing said sample for the level of any CCSA-2 antigen present therein; (C) correlating said level to at least one value predetermined for gauging the advancement of said cancer.

2. The method of claim 1, wherein said value is predetermined from an analysis of CCSA-2 level in non-cancerous tissue of said individual.

3. The method of claim 1, wherein said value is predetermined from one or more CCSA-2 levels from a person or from persons other than said individual.

4. The method of claim 1, wherein said correlating step identifies an individual with an advanced adenoma prior to malignant transformation.

5. A method for gauging efficacy of treatment for colorectal cancer, comprising: (A) obtaining first and second samples of blood from a subject suffering from said condition, wherein said samples are obtained at different points of said treatment; (B) determining a level of CCSA-2 for said first sample and said second sample, respectively; and then (C) comparing the level of CCSA-2 between each sample, whereby a difference in CCSA-2 level is an indicator of efficacy of said treatment.

6. The method of claim 5, wherein a lower level of CCSA-2 in said second sample indicates good efficacy of said treatment.

7. The method of claim 5, wherein said efficacy is a gauge of the survival of said subject suffering from a cancer condition.
